(19) United States
(12) Patent Application Publication
(10) Pub. No.: US 2022/0220428 A1
(43) Pub. Date: Jul. 14, 2022

(54) INCUBATION SYSTEM

(71) Applicant: EVORION BIOTECHNOLOGIES GMBH, Münster (DE)

(72) Inventors: Hans KLEINE-BRÜGGENEY, Münster (DE); Robert WEINGARTEN, Münster (DE); Sebastian BÜHREN, Nottuln (DE)

(21) Appl. No.: 17/611,354

(22) PCT Filed: May 15, 2020
(86) PCT No.: PCT/EP2020/063581

§ 371 Date: Nov. 15, 2021

(30) Foreign Application Priority Data
May 16, 2019 (DE) 10 2019 003 444.9

(51) Int. Cl. C12M 1/32; C12M 3/06; C12M 1/00; C12M 3/00; C12M 1/34

(52) U.S. Cl. CPC C12M 23/12; C12M 23/16; C12M 23/20; C12M 41/14; C12M 23/46; C12M 41/40; C12M 23/38

(57) ABSTRACT

The invention relates to an incubation system (26) comprising a preferably multilayered carrier (1) and a container (27). The carrier (1) is received in the container (27) in such a way that the container (27) and the carrier (1) are in fluid communication via at least one interface. Furthermore, the carrier (1) is fastened to the container (27) in such a way that the at least one interface for fluid exchange between the container (27) and the carrier (1) is tight.

The invention relates to an incubation system. Incubation systems are known, for example, from WO 2013/082612 A1. It describes an incubation system which has a carrier plate and a closure plate. The carrier plate is a cell culture plate having a plurality of reservoirs for receiving fluids. Furthermore, the known incubation system has a pneumatic device which generates a vacuum in the cell culture plate from above via the closure plate in order to keep the cell culture plate closed. The pneumatic device is also used to generate pressure in the reservoirs to move fluid contained therein. The disadvantage of such incubation systems is that they are not pressure-resistant, in particular due to the vacuum. A higher pressure results in the closure plate becoming detached from the cell culture plate and the incubation system becoming leaky.

The object of the invention is therefore to provide an incubation system that can be subjected to higher pressure and remains tight. The object is achieved by an incubation system according to claim 1. Advantageous embodiments are disclosed in the dependent claims, the description and the figures.

By providing a container in which the carrier is received in such a way that the container and the carrier are in fluid communication via at least one interface and furthermore the carrier is fastened to the plate in such a way that the at least one interface is tight for fluid exchange between the container and the carrier, a tight and pressure-resistant incubation system is created.

The invention is based on the basic idea of fastening the carrier to the container in such a way that corresponding interface openings, which are used for fluid exchange between carrier and container, cover one another and that there is no displacement of these interface openings, which can negatively affect the exchange of fluid between the container and the carrier.

In the context of the invention, “carrier” means a device having cavities for receiving fluids. In the context of the invention, a cavity can be, for example, a channel, a reservoir, or a chamber. The carrier is preferably plate-shaped. The carrier is particularly preferably multilayered. The carrier is, for example, a cell culture plate.

In the context of the invention, “container” means a device having cavities for receiving fluids. The cavities are preferably channels. Such a channel preferably extends from an outside of the container to an inside or underside of the container. The opening of the channel on the outside is used in particular to connect a line of a pneumatic device such as a control unit, which is described below. The opening of the channel on the underside or on the inside is used for fluid exchange between the container and the carrier. This is also described in more detail below.

According to the invention, the carrier is received in the container. In this context, “received” means that the carrier is at least partially arranged in the container. The container thus comprises a receiving space for at least partially accommodating the carrier. The carrier can, for example, be arranged completely in the receiving space. Alternatively, it is possible for the carrier to protrude from the receiving space.

The reception of the carrier in the container must be such that the container and the carrier are in fluid communication. In the context of the invention, the fact that a part (e.g. the carrier) is in fluid communication with another part (e.g. the container) means that a fluid exchange between the parts is possible. It can be a direct or indirect fluid exchange. In the case of the direct fluid exchange, the fluid can flow from one part directly into the other part. In the case of the indirect fluid exchange, the fluid can flow from one part into the other part only via at least one intermediate part (e.g. a distribution plate).

The carrier is fastened to the container in such a way that the interfaces for fluid exchange between the container and the carrier are tight. In this context, “tight” means that no fluid or only a negligibly small amount of fluid can escape at the interface. The interface is preferably tight for up to a fluid pressure of 2 bar. It is particularly preferred that all interfaces between the container and the carrier are tight.

In the context of the invention, “interface” means that point at the boundary between two parts that comprises an opening in one part through which the fluid can exit from the one part or can enter the one part (interface opening) and comprises an opening in the other part through which the fluid can exit from the other part or enter the other part (interface opening), the interface openings being arranged in such a way that a fluid exchange between the parts is possible via the interface openings. As an exception, the term “interface” does not fall under this definition if it is an electrical interface.

The carrier is fastened to the container in such a way that the at least one interface for fluid exchange between the container and the carrier is tight. In this context, “fastened” means that the carrier is connected to the container in such a way that no relative movement of carrier and container is possible or that only a slight relative movement is possible without the at least one interface for fluid exchange between the container and the carrier becoming leaky. This definition comprises the case where the container and the carrier are directly fastened. Therein, the container and the carrier touch one another. This definition also comprises the case that the carrier is fastened indirectly to the container. An intermediate part (e.g. a distribution plate) is arranged between the carrier and the container. In the latter case, the definition should be expanded to the extent that no relative movement of the carrier, container, and distribution plate is possible or that only a slight relative movement is possible without the at least one interface for fluid exchange between the container and the carrier becoming leaky.

The carrier can be fastened to the container by various types of connection. For example, a force fit, form fit, material fit, or a combination of two or three of these types of connection is conceivable. For example, the fastening can take place by means of a latching connection. A screw connection is also conceivable.

In a preferred embodiment, the carrier is fastened to the container by a clamping element. The carrier is arranged between the container and the clamping element in such a way that the plate is fastened to the container by a clamping force.

In a preferred embodiment, the incubation system has a cover for closing the container, the cover and the container forming an incubation chamber. The incubation system is preferably designed in such a way that, as a result of the closing of the container by the cover, cavities are created between the carrier and the cover. These cavities can be used, for example, for gas exchange. In a preferred embodiment, the cover has an opening (cover opening) which is designed and arranged in such a way that a region of the carrier in which, for example, cell cultures are located, can be exposed to light which enters the region from the outside through the cover opening. It is conceivable to close the cover opening with translucent material. For example, it can be glass or a light-filtering material. In a conceivable embodiment, the cover forms the clamping element.

In a preferred embodiment, the cover is in fluid communication with the container. Additionally or alternatively, the cover is in fluid communication with the carrier. The interfaces for fluid exchange between the cover and the container and/or between the cover and the carrier are preferably tight.

The carrier is preferably multilayered. In a preferred embodiment, the carrier has at least two layers which have interfaces for fluid exchange between the layers. In a preferred embodiment, the first layer is made of polymethyl methacrylate (PMMA). The first layer can have a microfluidic system. In a preferred embodiment, the second layer is made of polydimethylsiloxane (PDMS). The second layer preferably has a microfluidic system. The carrier preferably has a layer which is a glass plate. The glass plate preferably has a slide format (in particular, it is used as a slide). In a preferred embodiment, the glass plate stabilizes a flexible carrier structure (in particular a flexible structure of the first layer) and/or provides a planar surface. This can be important when using a microscope, for example, so that the Z focus does not change significantly. The glass plate is preferably the lowermost layer of the carrier.

In a preferred embodiment, the carrier has at least one reservoir. The carrier preferably has a plurality of reservoirs. A reservoir can be, for example, an input reservoir or an output reservoir. An input reservoir can be used, for example, to provide a fluid that is intended to be fed to a downstream region (for example a microfluidic system). An output reservoir can be used, for example, to receive fluids from upstream regions (such as the microfluidic system). They can be, for example, residual fluids that have to be removed from the system, such as trapped air. They can also be, for example, ready-prepared fluids that are to be analyzed. In a preferred embodiment, the carrier has 12 reservoirs. Of these, 8 input reservoirs and 4 output reservoirs are particularly preferred. A pressure difference can preferably be built up between the input and output reservoirs. This pressure difference can be both positive and negative. Thus, fluid can flow from the input reservoir to the output reservoir or from the output reservoir to the input reservoir. The input reservoirs and/or the output reservoirs can preferably be subjected to a pressure of up to 1 bar in order, for example, to force air from microfluidic channels through the carrier (preferably through the first layer).

Furthermore, the carrier can have at least one chamber. The chamber can be used, for example, for mixing fluids or for gas exchange. The chamber can be filled with a fluid, for example water.

In a preferred embodiment, the carrier is hermetically sealed and only accessible through corresponding input channels through which fluid can flow from the outside into the carrier, and output channels through which fluid can flow out of the carrier to the outside. In other words: If these channels are hermetically sealed, the carrier is also hermetically sealed to the outside and forms a hermetically sealed system.

In a preferred embodiment, the intermediate space between the carrier and the incubation chamber, in particular the space above the carrier, is completely sealed off from the outside and is preferably only connected to the external environment of the incubation chamber via at least one channel (preferably an input channel and also an output channel). This intermediate space, in particular the space above the carrier, preferably has increased humidity in order to prevent evaporation (e.g.: water evaporation) in the carrier and/or in the intermediate space. In a preferred embodiment, a humidified gas mixture, preferably five percent carbon dioxide, flows through the intermediate space, in particular the space above the carrier. The output channel can advantageously be connected to a gas analyzer in order to display changes in the gas composition during cell culture. Such devices can be, for example, GC-MS instruments.

In a preferred embodiment, the incubation system has a first connecting channel which connects the incubation chamber to the reservoir which is located in one layer, and a second connecting channel which connects the reservoir to another layer.

According to the invention, the container and the cover form the incubation chamber. The first connecting channel preferably connects the container to the reservoir. It is also conceivable that the first connecting channel connects the cover to the reservoir.

In a preferred embodiment, the reservoir has a reservoir opening. In this context, “reservoir opening” means an opening of the reservoir which is different from the opening that connects the reservoir to the first connecting channel, and which is also different from the opening that connects the reservoir to the second connecting channel.

In a preferred embodiment, the reservoir opening is attached to an outer surface of the carrier in such a way that the reservoir can be filled from the outside while the carrier is received in the container and is fastened to the container. This allows the container to be filled from the outside without having to loosen the fastening of the carrier to the container.

In a preferred embodiment, the incubation system has a reservoir closure for closing the reservoir opening. The reservoir closure can for example be the clamping element or a separate element which is arranged between the clamping element and the carrier. So that the fastening of the carrier to the container is maintained while the reservoir is being filled, the reservoir closure can, for example, be pushed onto the reservoir opening laterally between the clamping element and the carrier. It is also conceivable that the cover forms the reservoir closure. In a particularly preferred embodiment, the reservoir closure is such that repeated opening and closing of the reservoir opening is possible. The reservoir opening is preferably provided with a sealing element (e.g. an O-ring).

In a preferred embodiment, the incubation system has a partial closure for partially closing the reservoir opening, whereby a remaining opening remains which is closed by the reservoir closure. A sealing element is arranged between the partial closure and the reservoir closure in order to seal the remaining opening. The sealing element is preferably an O-ring. By partially closing the reservoir opening, the force that is required to seal the reservoir opening is reduced. The reservoir can thereby be sealed off more easily and more securely. The remaining opening preferably has an area of 2, 10, 50 or 100 mm².

In a preferred embodiment, the first connecting channel has an interface opening which is part of the interface between the incubation chamber and the layer in which the reservoir is located (reservoir layer). Furthermore, the second connecting channel has an interface opening which is part of the interface between the reservoir layer and the other layer. The interface openings are preferably on different planes. This can be advantageous to adapt the carrier according to the conditions of a microscope. For example, it is necessary to provide different planes so that a certain part of the carrier lies in the focus of a microscope. Alternatively, the interface openings are coplanar.

In a preferred embodiment, the interface opening of the second connecting channel and the reservoir are coaxial. In an alternative embodiment, the axis of the interface opening of the second connecting channel and the axis of the reservoir are different. This has the advantage that a channel in the other layer, which adjoins the second connecting channel, does not have to have an interface opening to the reservoir layer that is coaxial with the reservoir. The interface openings of the layers can therefore be chosen more flexibly.

In a preferred embodiment, the interface opening of the first connecting channel is closed by a closure element, wherein the closure element has a certain permeability. In this context, “permeability” means permeability for fluids. In this context, “certain” means that the permeability only exists for fluids of a certain property. An example of a property is the molecular size or the physical state of the fluid. The permeability can thus be selected such that, for example, air can penetrate the closure element, while the closure element is impermeable to liquids. In this way, liquids located in the reservoir can be protected against losses through evaporation and vaporization. At the same time, however, it is possible to transport these liquids to downstream points via the second connecting channel by, for example, feeding compressed air into the first connecting channel through the closure element. The compressed air can, for example, be enriched with carbon dioxide (e.g. 5 or 7 percent by volume). By evaporating less or no fluid, cell culture processes, for example, can be carried out for longer (e.g. for days). Likewise, the concentration of fluids does not change. In a preferred embodiment, the permeability is determined by the pore size of the closure element. The pore size is preferably 0.2, 1, 2 or 5 μm. In a preferred embodiment, the closure element is a membrane, which is preferably made of polytetrafluoroethylene.

In a preferred embodiment, the reservoir has a capacity of at most 200 μl. This is particularly advantageous in combination with a closure element which closes the interface of the first connecting channel. Such reservoirs make it possible to fill them with small amounts of fluids. This not only has the advantage of a smaller installation space, but also an economical use of fluids, which can be very expensive in the field of microfluidics. The risk of wasting expensive fluids through evaporation or vaporization is thereby averted or at least reduced.

In a preferred embodiment, the incubation system has a common cavity over at least two reservoirs, the reservoirs being connected to the same first connecting channel through the cavity. In this way, the same connecting channel can be used to subject a plurality of reservoirs to a pressure.

In a preferred embodiment, a first fluid and an anti-evaporation fluid to avoid evaporation of the first fluid are located in the reservoir, the fluids being immiscible and the anti-evaporation fluid having a lower density than the first fluid. Preferably, the fluids are liquid. As a result, the anti-evaporation fluid covers the first fluid in the reservoir and prevents it from evaporating or vaporizing. Preferably, the anti-evaporation fluid has a lower evaporation pressure. Preferably, the anti-evaporation fluid is a mineral oil or silicone oil. The anti-evaporation fluid is preferably biocompatible.

In a preferred embodiment, the incubation chamber has the dimensions of a 96-well plate, but at least the width and the length of the 96-well plate. This has the advantage that the incubation chamber has dimensions that are compatible with conventional microscopes.

In a preferred embodiment, the container has a first container channel, which is in fluid communication with the first connecting channel, and a second container channel. Furthermore, the carrier has a carrier channel which is in fluid communication with the second container channel, but not with the first connecting channel.

In the context of the invention, “container channel” means a channel that is located in the container. In the context of the invention, “carrier channel” means a channel that is located in the carrier. It is true that the first connecting channel is also a carrier channel. In the context of this embodiment, “carrier channel” means a channel that is located in the carrier but which differs from the first connecting channel.

In this way, a fluid system is created which has fluid paths that are independent of one another. This allows, for example, a reservoir to be opened in order to fill it without the atmospheric pressure also prevailing in the carrier channel. As a result, when the reservoir is being filled, the carrier channel can be subjected to a pressure in order, for example, to actuate valves in the carrier.

In a preferred embodiment, the first layer has partial layers and the reservoir is located in a partial layer. The partial closure for partially closing the reservoir opening is preferably located in a second partial layer. The first layer particularly preferably has a third partial layer which has a channel which is in fluid communication with the second connecting channel.

The second layer preferably also has partial layers. The second layer (which is preferably that layer which is referred to as “other layer” in other parts of the description) preferably has a thickness between 10 μm and 2 mm.

In a preferred embodiment, the second layer has a valve, which is preferably microfabricated, and the second container channel is used to actuate the valve. In this context, “actuation” means the opening or closing of the valve by controlling the fluid flow that is fed into the carrier channel via the second container channel. This has the advantage that the valve can be actuated even when the reservoir is open.

In a preferred embodiment, the incubation chamber comprises aluminum or an aluminum alloy. The high thermal conductivity of aluminum can be used to efficiently dissipate heat in the fluid system of the carrier from the fluid system. The use of another material that has a high thermal conductivity is also conceivable. It is possible for the cover, the container, or the cover and the container to comprise aluminum or an aluminum alloy.

In a preferred embodiment, the incubation chamber has at least one sensor. A temperature sensor or a humidity sensor, for example, can be considered as the sensor. In this context, “at least” means that the incubation chamber can have a plurality of sensors. This also comprises the case that the incubation chamber can have a plurality of different sensors, for example a temperature sensor and a humidity sensor.

In a preferred embodiment, the incubation chamber has at least one actuator. A heating element, a cooling element, or a light source, for example, can be considered as the actuator. In this context, “at least” means that the incubation chamber can have a plurality of actuators. This also comprises the case that the incubation chamber can have a plurality of different actuators, for example a heating element and a cooling element.

The heating element is, for example, a printed circuit board, also called a PCB. This has the advantage that such heating elements are inexpensive and also allow the integration of process control elements such as microprocessors and temperature sensors. The cooling element is, for example, a cooling channel. The light source is, for example, an LED, preferably a UV LED. It can be used, for example, to expose regions with cell cultures to the light of the light source. This is important, for example, in methods in which light-sensitive hydrogel matrices are used. The hydrogel gelling can then occur within the regions exposed to the light from the light source.

The at least one sensor and/or the at least one actuator can be attached to the cover, to the container, or to the cover and to the container. The sensor and/or the actuator is preferably attached on the side of the container and/or the cover, which side faces the carrier.

In a preferred embodiment, the incubation chamber has a printed circuit board which is preferably flexible. The printed circuit board can have the sensor, the actuator, and/or a memory unit. Due to the flexibility, the printed circuit board can be attached to the incubation chamber in such a way that it is located on different planes of the incubation chamber. This eliminates the need to use different printed circuit boards for different planes. The memory unit can, for example, be an erasable read-only memory such as EEPROM. The memory unit can be used, for example, to store the carrier's identification number. This is useful when different carriers are placed in the incubation chamber. In this way, the corresponding carrier can be identified by the read-only memory. A temperature sensor is preferably positioned in the carrier in such a way that it can measure the temperature at the position at which, for example, cells are cultivated. This position on the upper side of the slide is particularly preferred, the slide preferably being the glass plate, which particularly preferably forms the lowermost layer of the carrier.

In a preferred embodiment, the PCB and/or PCB components do not come into contact with the humidified gas in the intermediate space between the carrier and the incubation chamber—in particular above the carrier. This can be achieved by using appropriate sealing elements such as injected elastomers or sealing cables. The sealing elements can be attached to interfaces at which humidified gas can have access to the PCB/PCB components. The PCB/PCB components are preferably provided with an anti-fouling coating. In a preferred embodiment, the PCB/PCB components are arranged and coated with cast or injection-molded components such as silicone spray components, in particular thermally conductive spray components (e.g. Sylgard 160, DOWSIL™ EE-3200).

In a preferred embodiment, the incubation chamber has a distribution plate which connects the incubation chamber channels to carrier channels. In the context of the invention, “incubation chamber channel” means a channel that is located in the incubation chamber. It can be a container channel or a channel located in the cover (cover channel). The distribution plate preferably connects container channels to carrier channels.

According to the invention, the container is fastened to the carrier in such a way that the at least one interface for fluid exchange between the container and the carrier is tight. In the context of this embodiment, the container is fastened to the carrier in such a way that the at least two interfaces for fluid exchange between the container and the carrier are tight. One interface is used for the fluid exchange between the container and the distribution plate and the other interface for the fluid exchange between the distribution plate and the carrier. In a preferred embodiment, the distribution plate is part of the container. In a particularly preferred embodiment, the container and the distribution plate are designed in one piece.

The distribution plate is preferably made of PMMA. The distribution plate is preferably multilayered. The layers of the distribution plate can be connected to one another, for example, by diffusion bonding processes or solvent bonding processes.

In a preferred embodiment, the container has an electrical interface for checking the secure closing of the cover. For example, by means of the electrical interface, electrical contact is only established when the cover is closed as intended. Furthermore or alternatively, the container has a pneumatic interface for checking the secure closing of the cover. For example, low pressure or a pressure drop can be used to indicate that the cover was not closed or was accidentally opened.

In a preferred embodiment, the container has an electrical interface for checking the precise fit of the carrier into the container. Furthermore or alternatively, the container has a pneumatic interface for checking the precise fit of the carrier into the container.

In a preferred embodiment, the incubation system has a control unit for regulating variables in the incubation chamber and the carrier, wherein the control unit is connected electrically, pneumatically, and/or hydraulically to the incubation chamber and is in fluid communication with the incubation chamber and the carrier. In this case, the regulation takes place in particular by means of the at least one sensor and the at least one actuator.

In the context of the invention, the term “regulating” comprises the case that variables are regulated, the case that variables are controlled, and the case that variables are regulated and other variables are controlled. The first case is preferred.

According to this embodiment, the control unit is pneumatically and/or hydraulically connected to the incubation chamber. This is to be understood as meaning that the control unit has at least one line via which the control unit can introduce fluid into the at least one incubation chamber channel (e.g. container channel). In a preferred embodiment, a plurality of lines of the control unit are connected to a plurality of incubation chamber channels (e.g. container channels). In each case, one line is particularly preferably connected to an incubation chamber channel. The control unit is particularly preferably designed in such a way that the introduction modalities for each incubation chamber channel (e.g. container channel) are individually determined. The modality of introduction is to be understood as fluid properties (see below) and the fact whether a fluid is being introduced.

According to this embodiment, the control unit is used to regulate variables in the incubation chamber and the carrier. In this context, “variable” means a physical variable. It can be a state variable or a process variable. For example, the temperature, the fluid property, or the humidity come into consideration as a variable. A fluid property is, for example, the type of fluid, the pressure of the fluid, the fluid direction, the flow rate of the fluid and the fluid composition.

The incubation system can be used to regulate microfluidic processes. Such processes can comprise encapsulating preferably individual cells in hydrogel matrices, among other things. Another example is the demulsification of generated cell-charged hydrogel matrices. Furthermore, the cells can be positioned at a fixed and predetermined location by means of the incubation system. It is also possible to create suitable cell culture conditions, such as supplying certain regions with a certain fluid. Fluid flows can also be regulated. In addition, process sequences can be programmed so that they can run repeatedly.

In a preferred embodiment, the control unit has a supply of at least one fluid which can be supplied to the carrier via an interface for fluid exchange between the control unit and the carrier. The fluid is preferably a cooling liquid. The cooling liquid is preferably intended to be fed to a cooling channel in the cover or the container in order to effect cooling of the carrier. In an alternative embodiment, the incubation system has a supply which is connected to the control unit. In this case, the supply is not part of the control unit but is connected to it in order to supply it with the at least one fluid.

The invention is explained below with reference to figures. The figures show embodiments of the invention only by way of example. In the drawings:

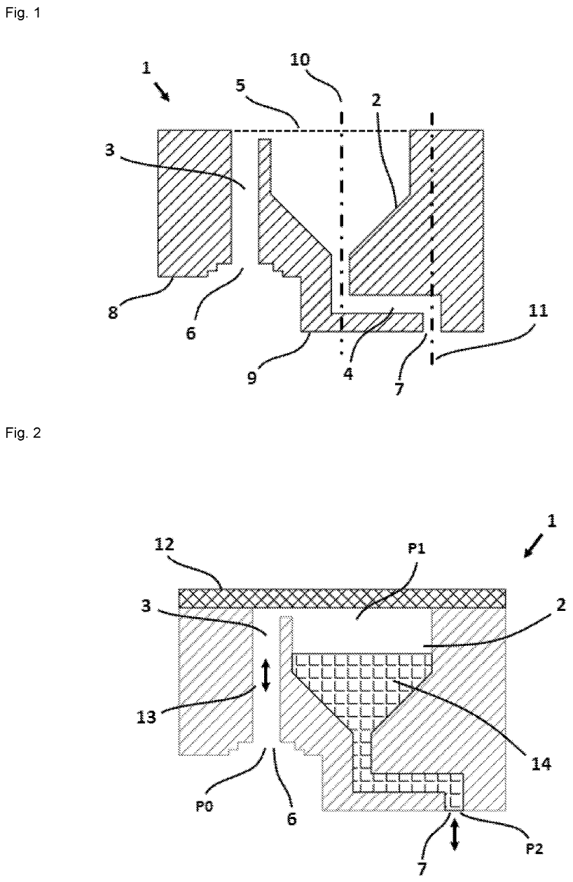

FIG. 1 is a section of a carrier in the sectional view,

FIG. 2 shows a carrier according to FIG. 1, which is supplemented by further elements,

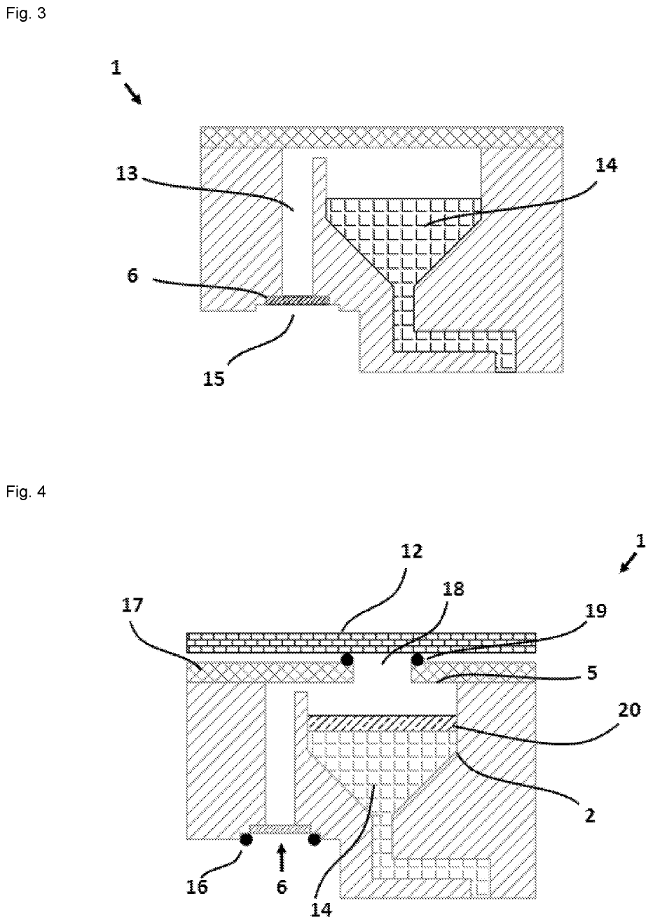

FIG. 3 shows a carrier according to FIG. 1, which is supplemented by a closure element,

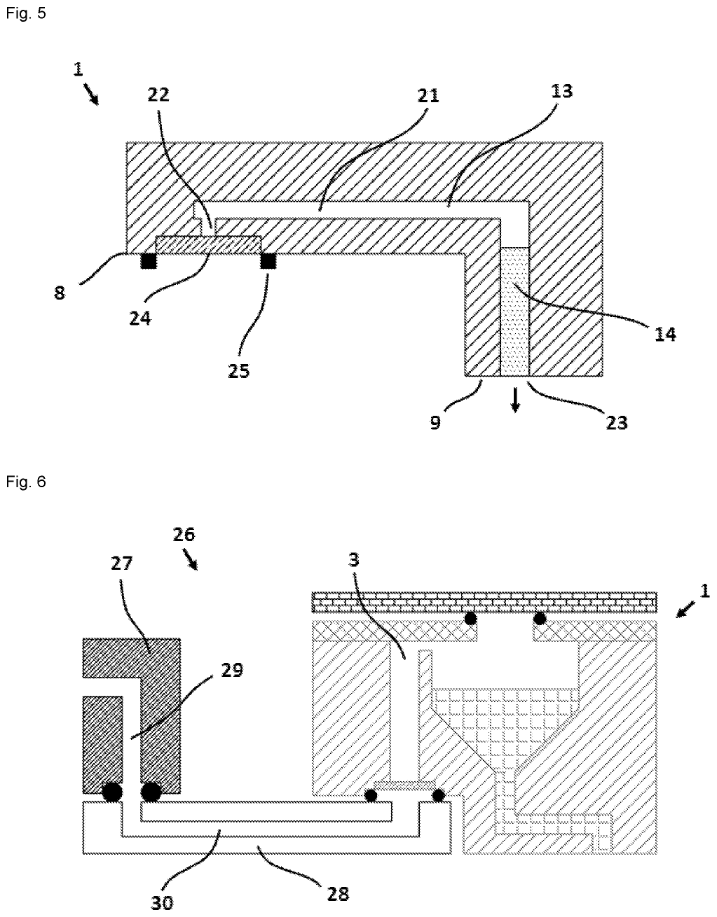

FIG. 4 shows a carrier according to FIG. 1, which is supplemented by further elements,

FIG. 5 is another section of a carrier in the sectional view,

FIG. 6 is a section of an incubation system in the sectional view,

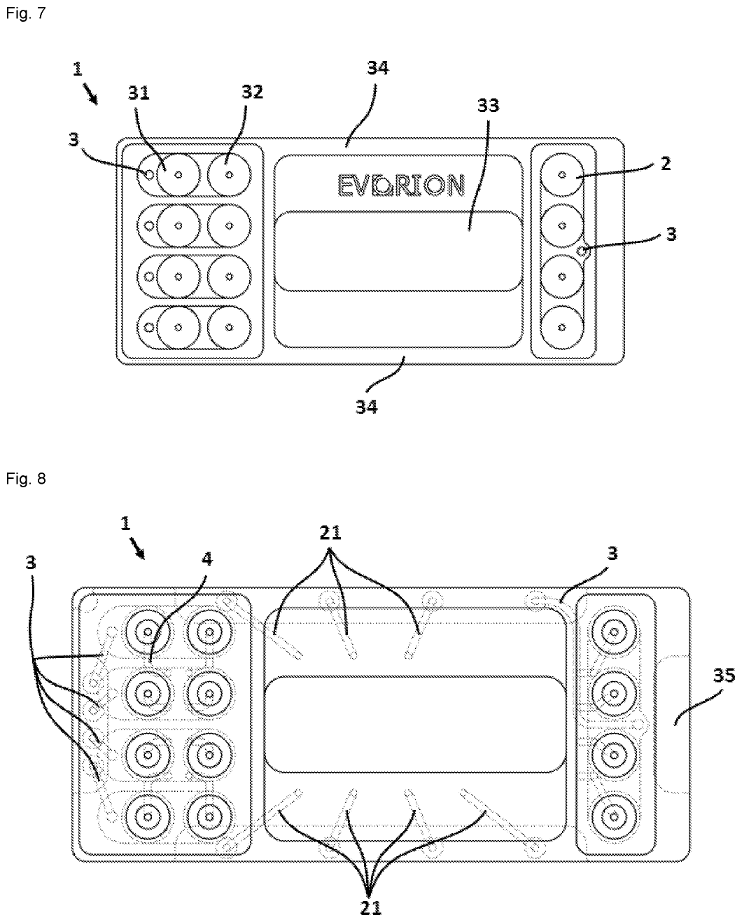

FIG. 7 shows a carrier in top view,

FIG. 8 shows the carrier according to FIG. 7, in which further planes are visible,

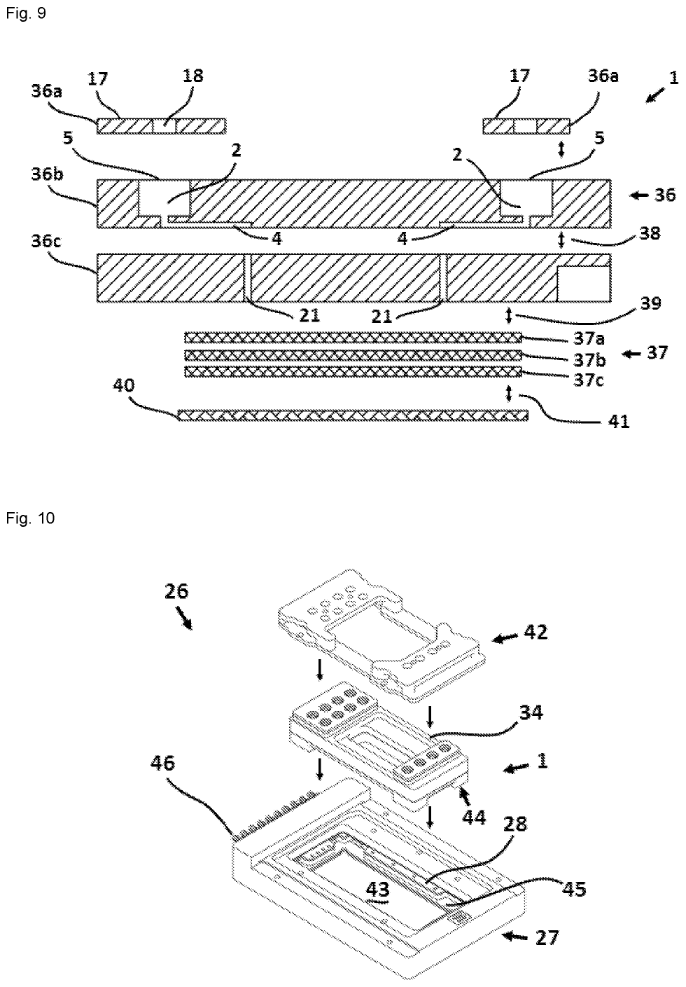

FIG. 9 shows a carrier in the sectional view,

FIG. 10 is an exploded view of an incubation system,

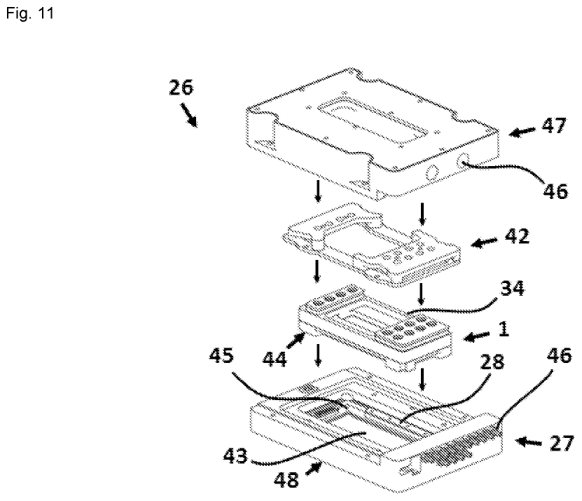

FIG. 11 is an exploded view of an incubation system.

FIG. 1 shows an embodiment of a carrier 1 in the sectional view. A part of the carrier 1 in which the reservoir 2 is located can be seen. In addition to the reservoir 2, the carrier 1 comprises a first connecting channel 3 which is used to connect the incubation chamber to the reservoir 2. As will be explained later with reference to FIG. 6, the carrier is arranged in a container and the container is in fluid communication with the carrier. A container channel is connected to the first connecting channel via a distribution channel. The carrier further comprises a second connecting channel 4 which is intended to connect the reservoir 2 to another layer. The reservoir 2 has a reservoir opening 5. The first connecting channel 3 has an interface opening 6 which is part of the interface between the incubation chamber and the reservoir layer (first interface opening). Furthermore, the second connecting channel has an interface opening 7 which is part of an interface between the reservoir layer and the other layer (second interface opening). The first interface opening 6 lies on a first plane 8 and the second interface opening 7 lies on a second plane 9. The first plane 8 and the second plane 9 are different planes. The axis 10 of the reservoir and the axis 11 of the second interface opening are different. They are parallel.

FIG. 2 shows the carrier 1 according to FIG. 1, which is supplemented by further elements. The carrier 1 thus has a reservoir closure 12 which allows the reservoir 2 to be opened and closed repeatedly. A fluid 13 of a first type (primary fluid) can be introduced into the reservoir 2 via the first interface opening 6 and the first connecting channel 3. The left double arrow indicates that the primary fluid 13 can flow in both directions. A fluid 14 of a second type (secondary fluid) is located in the reservoir 2. The right double arrow also indicates the possible flow directions here. The direction in which the secondary fluid 14 flows can be determined by adjusting the pressure in the reservoir through the primary fluid 13. If, for example, the pressure P1 is greater than the pressure P2, the secondary fluid 14 flows out of the reservoir 2 via the second interface opening 7.

FIG. 3 shows the carrier 1 from FIG. 1, which is supplemented by a closure element 15. The closure element closes the first interface opening 6. It has a certain permeability that allows the primary fluid 13 to pass through, but prevents the secondary fluid 14 from passing through. In this way, the loss of part of the secondary fluid 14 through vaporization thereof is prevented.

FIG. 4 shows the carrier 1 from FIG. 1, which is supplemented by further elements. On the one hand, the first interface opening 6 is provided with a sealing element 16. The sealing element 16 is an O-ring. Furthermore, the reservoir 2 (as in FIG. 2) is provided with a reservoir closure 12. In contrast to FIG. 2, the carrier 1 has a partial closure 17 for partially closing the reservoir opening 5, whereby a remaining opening 18 remains. A sealing element 19 is arranged between the reservoir closure 12 and partial closure 17 in the region around the remaining opening. The sealing element 19 is an O-ring. This closure construction has the advantage that lower sealing forces have to be applied and that the reservoir opening 5 can be sealed more securely. Furthermore, an anti-evaporation fluid 20 is located in the reservoir 2 to avoid evaporation of the secondary fluid 14. The anti-evaporation fluid 20 has a lower density than the secondary fluid 14 and is immiscible therewith.

FIG. 5 shows another part of the carrier 1 in which a carrier channel 21 is located. The carrier channel 21 is connected to a container channel via a distribution channel. However, the carrier channel 21 is not in fluid communication with the first connecting channel 3 (see, for example, FIG. 1). Thus, the carrier channel 21 is also not in fluid communication with the container channel, which is connected to the first connecting channel via a distribution channel. The carrier channel 21 has an interface opening 22 which is part of the interface between the container and the layer in which the carrier channel is located (carrier channel layer) (third interface opening). Furthermore, the carrier channel 21 has an interface opening 23 which is part of the interface between the carrier channel layer and another layer (fourth interface opening). The third interface opening 22 is closed with a closure element 24 which has a certain permeability. The permeability is such that the closure element 24 allows the passage of a primary fluid 13, but prevents the passage of a secondary fluid 14. A sealing element 25 can be arranged between the carrier channel layer and the distribution plate in order to seal the carrier channel 21. The secondary fluid 14 is located in the carrier channel 21. By introducing the primary fluid 13 via the third interface opening 22 into the carrier channel 21, a pressure can be built up which results in the secondary fluid 14 being moved in the direction of the fourth interface opening 23 and emerging from it. As a result, the secondary fluid 14 can get into another layer of the carrier 1 in order, for example, to actuate (for example to close) a valve in the other layer. A corresponding movement of the primary fluid 13 and thus of the secondary fluid 14 in the opposite direction is also possible (e.g. to open the valve). The third interface opening 22 lies on the first plane 8 and the fourth interface opening 23 lies on the second plane 9.

FIG. 6 shows a section of an exemplary incubation system 26 in the sectional view. The incubation system 26 has a carrier 1 according to FIG. 4, a container 27, and a distribution plate 28. The container 27 is made of aluminum. One can see, among other things, a container channel 29 and a distribution channel 30, which are in fluid communication with the first connecting channel 3.

FIG. 7 shows an exemplary carrier 1 in top view. The sections of the carrier in the previous figures can be part of the carrier 1 according to FIG. 7. The carrier 1 has eight reservoirs on the left. The four left reservoirs of the eight reservoirs are filled with a first reservoir fluid 31 and the four right reservoirs of the eight reservoirs are filled with a second reservoir fluid 32. In pairs, two reservoirs have a common first connecting channel 3. A chamber 33 is located in the center. The chamber 33 is preferably filled with water. Four reservoirs 2 are located on the right side, which reservoirs are output reservoirs. They are all connected via a common connecting channel 3. In order to fasten the carrier 1 to the container in such a way that the at least one interface for fluid exchange between the container and the carrier is tight, a clamping surface 34 is provided. A clamping force can be exerted on the clamping surface via a clamping element, which leads to the carrier being fastened to the container according to the invention.

FIG. 8 shows further elements of the carrier 1, which are shown in dashed lines because they are located in deeper planes of the carrier 1 that are not visible from the outside. Four first connecting channels 3 can be seen on the left. Each first connecting channel 3 is provided to be in fluid communication with a respective container channel 27 (see FIG. 6). Further carrier channels 21 can be seen above and below. Of these, seven carrier channels are valve actuation channels, i.e. they are used to actuate valves. The eighth carrier channel 21 is a common first connecting channel 3 for the output reservoirs, i.e. it is used to subject the four right reservoirs simultaneously to fluid pressure. On the far right, there is a position 35 for an electrical interface. The electrical interface can be used, for example, to check whether the carrier 1 is inserted with an accurate fit in the container.

FIG. 9 shows an embodiment of the carrier 1 in the sectional view. The carrier 1 comprises an upper layer 36, a lower layer 37, and a lowermost layer 40. The upper layer comprises partial layers 36a, 36b and 36c, which consist of PMMA and which are connected by means of solvent 38. This is also known as solvent bonding. The upper partial layer 36a has two parts which each form partial closures 17 for partially closing the reservoir openings 5. Two reservoirs 2 are visible, which are located in the middle partial layer 36b. The partial layer 36b further comprises second connecting channels 4. The first connecting channels are not visible in this sectional view. Furthermore, the upper layer 36 comprises a lower partial layer 36c, which has carrier channels which connect the second connecting channels and channels in the lower layer 37 (not visible). The lower layer 37 also comprises three partial layers 37a, 37b and 37c. It forms a multilayered microfluidic system that consists of PDMS. The upper layer and the lower layer are connected via oxygen plasma and a solvent 39. The lower layer 37 is connected to the lowermost view 40, which is preferably a glass plate, via oxygen plasma 41. The glass plate has a thickness between 0.1 and 1 mm.

FIG. 10 shows an exploded view of an exemplary incubation system 26. The container 27, the carrier 1, and the clamping element 42 are shown. The carrier 1 can, for example, be the carrier from the previous figures (in particular FIG. 7). The distribution plate 28 is located in the container 27. The bottom of the container 27 is open. The container thus has a bottom opening 43. A microscope can be used, for example, to look into the incubation chamber from the outside through the bottom opening 43. In a preferred embodiment of the incubation system (which is not restricted to the exemplary figures), the underside 44 of the carrier is located on the bottom 45 of the container. Furthermore, preferably (and not restricted to the exemplary figures), the underside of the carrier, which is preferably the underside of the glass plate, lies on the same plane as the underside 48 of the container. The container 27 also has connections 46 for lines (not shown) of the control unit. Ten such connections 46 can be seen. The four vertical and downward arrows indicate a preferred sequence of installation steps: First, the carrier 1 is inserted into the container 27 and then the clamping element 42 is positioned on the carrier 1. The clamping element 42 is fastened to the container 27, for example by means of screws (not shown), so that the carrier 1 is clamped between the clamping element 42 and the container 27. As a result, the carrier 1 is fastened to the container 27 in such a way that the interfaces for fluid exchange between the container (27) and the carrier (1) are tight. The clamping element 42 exerts a clamping force on the clamping surface 34 of the carrier. In this embodiment, the clamping element 42 can be viewed as a cover which, together with the container, forms the incubation chamber.

FIG. 11 shows an exploded view of an incubation system which differs from that from FIG. 10 in that it has a cover 47 which is not identical to the clamping element 42. The cover 47, together with the container 27, forms the incubation chamber within which the carrier 1 is arranged. Furthermore, FIG. 11 shows the incubation system 26 from the other side than FIG. 10. Fourteen connections 46 for lines of the control unit, which are arranged on the container 27, are therefore shown. Furthermore, two connections 46 for lines of the control unit on the cover 47 are visible.

### LIST OF REFERENCE SIGNS

1 Carrier
2 Reservoir
3 First connecting channel
4 Second connecting channel
5 Reservoir opening
6 First interface opening
7 Second interface opening
8 First plane
9 Second plane
10 Axis of the reservoir
11 Axis of the second interface opening
12 Reservoir closure
13 Primary fluid
14 Secondary fluid
15 Closure element
16 Sealing element
17 Partial closure
18 Remaining opening
19 Sealing element (reservoir opening)
20 Anti-evaporation fluid
21 Carrier channel
22 Third interface opening
23 Fourth interface opening
24 Closure element (third interface opening)
25 Sealing element (third interface opening)
26 Incubation system
27 Container
28 Distribution plate
29 Container channel
30 Distribution channel
31 First reservoir fluid
32 Second reservoir fluid
33 Chamber
34 Clamping surface
35 Position for electrical interface
36 Upper layer of the carrier
36a Upper partial layer
36b Middle partial layer
36c Lower partial layer
37 Lower layer of the carrier
37a Upper partial layer
37b Middle partial layer
37c Lower partial layer
38 Solvent
39 Oxygen and solvents
40 Glass plate
41 Oxygen plasma
42 Clamping element
43 Container bottom opening
44 Carrier underside
45 Container bottom
46 Connection for lines of the control unit
47 Cover
48 Underside of the container

1. Incubation system (26) comprising
a preferably multilayered carrier (1),
a container (27)
wherein the carrier (1) is received in the container (27) in such a way that the container (27) and the carrier (1) are in fluid communication via at least one interface and
the carrier (1) is fastened to the container (27) in such a way that the at least one interface for fluid exchange between the container (27) and the carrier (1) is tight.

2. Incubation system (26) according to claim 1, comprising a clamping element (42), wherein the carrier (1) is arranged between the container (27) and the clamping element in such a way that the carrier (1) is fastened to the container (27) by a clamping force.

3. Incubation system (26) according to either claim 1 or claim 2, comprising a cover for closing the container (27), wherein the cover forms an incubation chamber with the container (27),
wherein the incubation chamber preferably has the dimensions of a 96-well plate, but at least the width and length of the 96-well plate,
wherein the cover is preferably in fluid communication with the container (27) and/or with the carrier (1), wherein, preferably, the interfaces for fluid exchange between the cover and the container (27) and/or between the cover and the carrier (1) are tight.

4. Incubation system (26) according to any of the preceding claims, wherein one layer (36) has an interface opening which is arranged at the boundary with another layer (37) and allows the fluid exchange between the layers.

5. Incubation system (26) according to any of the preceding claims, wherein the carrier (1) has at least one reservoir (2).

6. Incubation system (26) according to the preceding claim, comprising a first connecting channel (3) which connects the incubation chamber to the reservoir (2) which is located in one layer (36), and a second connecting channel (4) which connects the reservoir (2) to another layer (37).

7. Incubation system (26) according to either claim 6 or claim 7, wherein the reservoir (2) has a reservoir opening (5) and preferably a reservoir closure (12) for closing the reservoir opening (5), which reservoir closure is in particular such that repeated opening and closing is possible.

8. Incubation system (26) according to the preceding claim, comprising a partial closure (17) for partially closing the reservoir opening (5), whereby a remaining opening (18) remains which is closed by the reservoir closure (12) and wherein a sealing element (19), preferably an O-ring, is arranged between the partial closure (17) and the reservoir closure (12) in order to seal the remaining opening (18).

9. Incubation system (26) according to any of claims 6 to 8, wherein the first connecting channel (3) has an interface opening (6) which is part of the interface between the incubation chamber and the layer (36) in which the reservoir (2) is located, and the second connecting channel (4) has an interface opening (7) which is part of an interface between the layer (36) in which the reservoir (2) is located and the other layer (37), wherein the interface openings (6, 7) are on different planes (8, 9).

10. Incubation system (26) according to any of claims 6 to 9, wherein the interface opening of the first connecting channel (3) is closed by a closure element (15), wherein the closure element (15) has a certain permeability.

11. Incubation system (26) according to any of the preceding claims 6 to 10, wherein the container (27) has a first container channel (29), which is in fluid communication with the first connecting channel (3), and a second container channel, and the carrier (1) has one carrier channel (21) which is in fluid communication with the second container channel but not with the first connecting channel (3).

12. Incubation system (26) according to any of the preceding claims, wherein the incubation chamber has at least one sensor, for example a temperature sensor or a humidity sensor, and/or an actuator, preferably a heating element, a cooling element, or a light source.

13. Incubation system (26) according to any of the preceding claims, wherein the incubation chamber, in particular the container (2), has a distribution plate (28) which connects the incubation chamber channels, preferably the container channels (29), to carrier channels (3, 4, 21) via distribution plate channels (28).

14. Incubation system (26) according to any of the preceding claims, wherein the container (27) has an electrical and/or pneumatic interface for checking the secure closing of the cover and/or has an electrical interface for checking the precise fit of the carrier (1) into the container (27).

15. Incubation system (26) according to any of the preceding claims, comprising a control unit for regulating variables in the incubation chamber and the carrier (1), wherein the control unit is connected electrically, pneumatically, and/or hydraulically to the incubation chamber and is in fluid communication with the incubation chamber and the carrier (1), wherein the regulation takes place in particular by means of the at least one sensor and the at least one actuator.
